(19) United States
(12) Patent Application Publication
(10) Pub. No.: US 2014/0194827 A1
(43) Pub. Date: Jul. 10, 2014

(54) DISPOSABLE PRE-FILLED SYRINGE

(71) Applicants: Pei-Hsin Hsu, Taichung City (TW); Pei-Yang Hsu, Taichung City (TW); Wei-Ni Hsu, Taichung City (TW)

(72) Inventors: Pei-Hsin Hsu, Taichung City (TW); Pei-Yang Hsu, Taichung City (TW)

(73) Assignees: Pei-Hsin Hsu, Taichung City (TW); Wei-Ni Hsu, Taichung City (TW); Pei-Yang Hsu, Taichung City (TW)

(21) Appl. No.: 14/064,530

(22) Filed: Oct. 28, 2013

(30) Foreign Application Priority Data
Jan. 9, 2013 (TW) 102100689

(51) Int. Cl. A61M 5/32

(52) U.S. Cl. CPC A61M 5/3202
USPC 604/192

(57) ABSTRACT

A disposable pre-filled syringe has a protecting tube, a medication filling tube, a separating plug, a pushing module, and a needle module. The medication filling tube is made of material stable in chemical property and is mounted in the protecting tube. The separating plug is mounted in an end of the medication filling tube. The pushing module is slidably inserted into the medication filling tube opposite to the separating plug. The needle module is mounted on an end of the protecting tube.

### BACKGROUND OF THE INVENTION

1. Field of the Invention

The present invention relates to a disposable syringe, and more particularly to a disposable pre-filled syringe.

2. Description of Related Art

A conventional disposable syringe is made by plastic injection molding, such that the conventional disposable syringe is low-cost and for single use only. After a disposable syringe is used, the syringe has to be disposed of or discarded to prevent infections among users.

Prior to use, the conventional disposable syringe is filled with medication which is prepared from a preserved bottle or vial. Such a handling process is complicated and may produce many medical wastes. On the other hand, the conventional plastic disposable syringe is not suitable for using as a pre-filled syringe since the plastic ingredients and additives may spontaneously leach into, and/or be extracted out by the pre-filled medication, specifically when referring to the biopharmaceuticals. Moreover, some currently available disposable pre-filled syringes are used to store high value medications, such as vaccines or drugs for treating chronic diseases, and are made of special thermoformed polymer or glass materials. As such, the costs of those conventional disposable pre-filled syringes are high, and the manufacturing processes of those syringes are complicated and energy-consuming.

### SUMMARY OF THE INVENTION

The main objective of the present invention is to provide a disposable pre-filled syringe to resolve the aforementioned problems.

The disposable pre-filled syringe comprises a protecting tube, a medication filling tube, a separating plug, a pushing module, and a needle module.

The protecting tube comprises an axis, an outside wall, an inside wall, a holding portion, a needle mounting portion, a tube space, and an abutting portion. The outside wall is annularly formed along the axis of the protecting tube and is located at an exterior of the protecting tube. The inside wall is annularly formed along the axis of the protecting tube and is located at an interior of the protecting tube. The holding portion comprises an insertion hole formed through the holding portion. The needle mounting portion is opposite to the holding portion. The tube space is formed in the protecting tube along the axis of the protecting tube and communicates with the insertion hole. The abutting portion is mounted radially on the inside wall and is adjacent to the needle mounting portion.

The medication filling tube is transparent and made of material stable in chemical property, is mounted in the tube space and comprises an axis, a medication storage space, a pushing module entrance, and a plug hole. The axis of the medication filling tube is parallel with the axis of the protecting tube. The medication storage space is formed in the medication filling tube along the axis of the medication filling tube. The pushing module entrance is formed through an end of the medication filling tube and communicates with the medication storage space. The plug hole is formed through an end of the medication filling tube opposite to the pushing module entrance and communicates with the medication storage space.

The separating plug is mounted into the plug hole and abuts the abutting portion. The pushing module is slidably and hermetically mounted in the medication filling tube and comprises a pushing stick and a pushing plug. The pushing stick is mounted into the medication storage space through the pushing module entrance and has a combining portion. The pushing plug is removably mounted on the combining portion and slidably and hermetically mounted in the medication storage space. The needle module is mounted on the needle mounting portion.

Other objectives, advantages and novel features of the present invention will become more apparent from the following detailed description when taken in conjunction with the accompanying drawings.

### BRIEF DESCRIPTION OF THE DRAWINGS

FIG. 1 is an exploded side view in partial section of a first preferred embodiment of a disposable pre-filled syringe in accordance with the present invention;

FIG. 2 is a side view in partial section of the disposable pre-filled syringe in FIG. 1;

FIG. 3 is an enlarged side view of the disposable pre-filled syringe in FIG. 2;

FIG. 4 is a cross sectional side view of the disposable pre-filled syringe along line 4-4 in FIG. 2;

FIG. 5 is an enlarged side view of the disposable pre-filled syringe in FIG. 4;

FIG. 6 is an operational side view of the disposable pre-filled syringe in FIG. 1;

FIG. 7 is an exploded side view in partial section of a second preferred embodiment of the disposable pre-filled syringe of the present invention;

FIG. 8 is an operational side view in partial section of the disposable pre-filled syringe in FIG. 7;

FIG. 9 is an operational side view in partial section of the disposable pre-filled syringe in FIG. 7;

FIG. 10 is an operational side view in partial section of the disposable pre-filled syringe in FIG. 7;

FIG. 11 is an exploded side view in partial section of a third preferred embodiment of the disposable pre-filled syringe of the present invention;

FIG. 12 shows operational side views in partial section of the disposable pre-filled syringe in FIG. 11;

FIG. 13 is an exploded side view in partial section of a fourth preferred embodiment of the disposable pre-filled syringe of the present invention;

FIG. 14 is an operational side view in partial section of the disposable pre-filled syringe in FIG. 13;

FIG. 15 is an operational side view in partial section of a fifth preferred embodiment of the disposable pre-filled syringe of the present invention;

FIG. 16 is an enlarged side view of the disposable pre-filled syringe in FIG. 15;

FIG. 17 is an operational side view in partial section of a sixth preferred embodiment of the disposable pre-filled syringe of the present invention;

FIG. 18 is an enlarged side view of the disposable pre-filled syringe in FIG. 17;

FIG. 19 is an operational side view in partial section of a seventh preferred embodiment of the disposable pre-filled syringe of the present invention; and

FIG. 20 is an operational side view in partial section of an eighth preferred embodiment of the disposable pre-filled syringe of the present invention.

### DETAILED DESCRIPTION OF PREFERRED EMBODIMENT

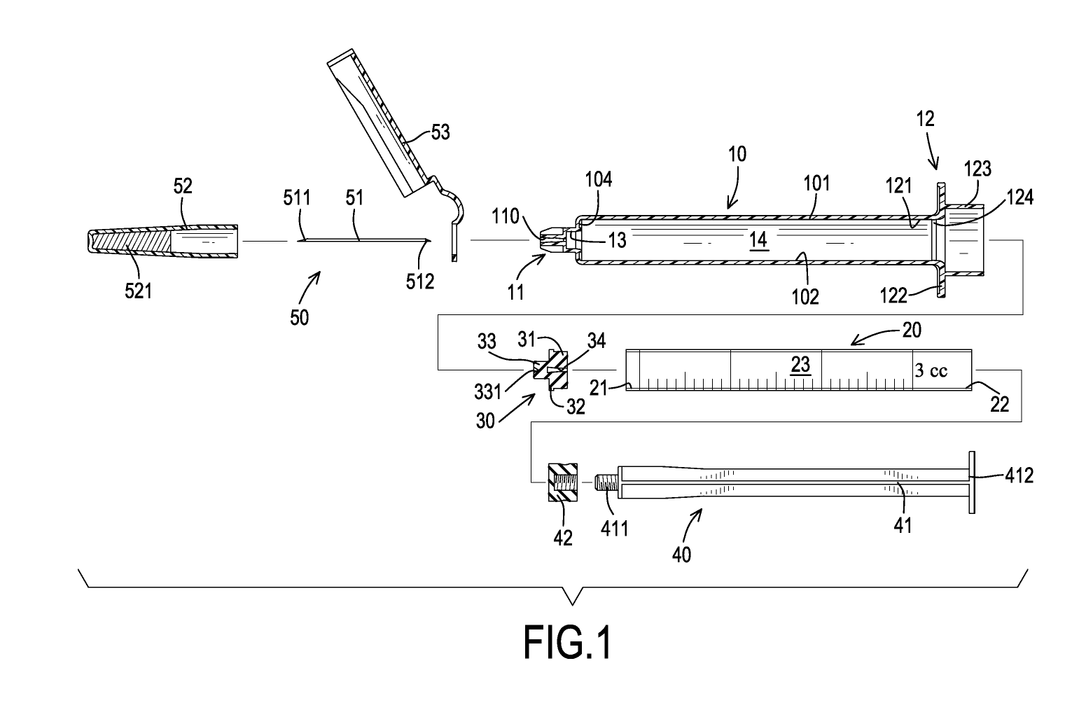

With reference to FIGS. 1 to 6, a first preferred embodiment of a disposable pre-filled syringe in accordance with the present invention comprises a protecting tube 10, a medication filling tube 20, a separating plug 30, a pushing module 40, and a needle module 50.

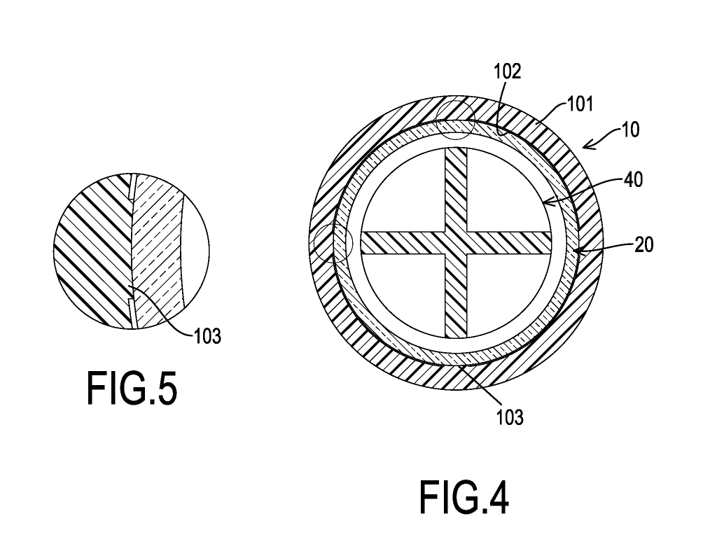

With reference to FIGS. 1 to 5, the protecting tube 10 may be made of plastic and comprises an axis, an outside wall 101, an inside wall 102, a tube space 14, multiple ribs 103, an abutting portion 104, a holding portion 12, a trough 13, and a needle mounting portion 11. The outside wall 101 and the inside wall 102 are annularly formed along the axis of the protecting tube 10 and are located respectively at an exterior and an interior of the protecting tube 10. With reference to FIGS. 4 and 5, the ribs 103 longitudinally protrude from the inside wall 102. The ribs 103 are arranged at equal spacing intervals.

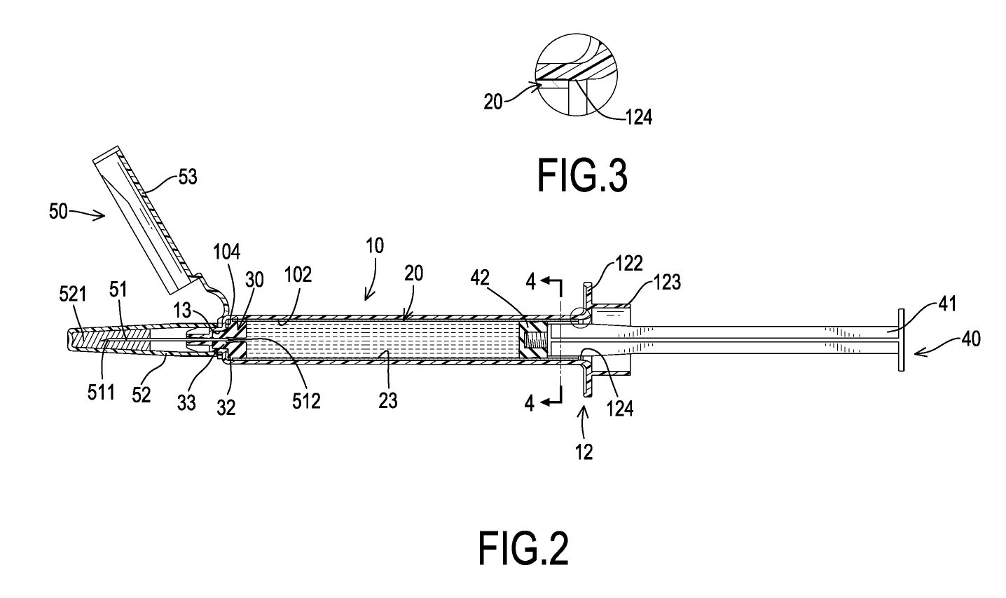

With reference to FIGS. 1 and 2, the abutting portion 104 is mounted radially on the inside wall 102 and is adjacent to a front end of the inside wall 102. The tube space 14 is formed in the protecting tube 10 along the axis of the protecting tube 10. The holding portion 12 is formed at a rear end of the protecting tube 10 and comprises an insertion hole 121, a holding flange 122, an annular piece 123, and an engaging edge 124. The insertion hole 121 is formed through the holding portion 12 and communicates with the tube space 14. The holding flange 122 radially and partial-annularly protrudes from an exterior of the holding portion 12. The annular piece 123 is mounted on a rear side of the holding flange 122. The engaging edge 124 circumferentially protrudes from an interior of the holding portion 12 and is adjacent to the insertion hole 121. The trough 13 is tubular, is connected with the abutting portion 104, is located at a front end of the protecting tube 10 and communicates with the tube space 14. The needle mounting portion 11 is mounted around and abuts the front end of the inside wall 102. The needle mounting portion 11 comprises a needle mounting hole 110 formed through the needle mounting portion 11 along the axis of the protecting tube 10 and communicating with the tube space 14.

With reference to FIGS. 1 and 2, the medication filling tube 20 is used to be filled with medication. The medication filling tube 20 may be removably mounted in the tube space 14. Alternatively, the medication filling tube 20 may be fixed in the tube space 14 as an integral part of the protecting tube 10. The medication filling tube 20 may be made of transparent and thermoplastic material, such as glass or cyclic olefin polymers, which is stable in chemical property and hardly releases ingredients or additives into the pre-filled medication. The medication filling tube 20 comprises a medication storage space 23, a pushing module entrance 22, and a plug hole 21. The medication storage space 23 is formed in the medication filling tube 20 along an axis of the medication filling tube 20. The pushing module entrance 22 and the plug hole 21 are formed through two opposite ends of the medication filling tube 20 respectively and communicate with the medication storage space 23. The pushing module entrance 22 aligns with the insertion hole 121. The plug hole 21 aligns with the trough 13. An exterior of the medication filling tube 20 abuts the ribs 103. The two ends of the medication filling tube 20 abuts the engaging edge 124 and the abutting portion 104 respectively, such that the position of the medication filling tube 20 relative to the protecting tube 10 is fixed by the engaging edge 124 and the abutting portion 104. The medication filling tube 20 may have a constant diameter along the axis of the medication filling tube 20. Alternatively, the medication filling tube 20 may be tapered toward the plug hole 21.

With reference to FIGS. 1 and 2, the separating plug 30 is mounted in the plug hole 21 and the trough 13. The separating plug 30 is made of chemically inert thermoplasctic elastomer that is stable in chemical property. The separating plug 30 comprises a plug body 31, a block flange 32, a protrusion 33, a puncture slit 331, and a leading hole 34. The plug body 31 is mounted in the plug hole 21 hermetically. The block flange 32 is annularly formed around the plug body 31, is adjacent to the front end of the plug body 31 and abuts the abutting portion 104, such that the block flange 32 is used as a washer for the medication filling tube 20. The protrusion 33 is formed on and protrudes from a front end of the plug body 31 and is mounted in the trough 13. The puncture slit 331 is formed on the front end of the protrusion 33. The leading hole 34 is formed in the separating plug 30 with an axis in accordance with the axis of the protecting tube 10 and communicates with the medication storage space 23. The puncture slit 331 and the leading hole 34 align with the needle mounting hole 110, and a needle can penetrate through and be fixed in the needle mounting hole 110. The needle can further penetrate the protrusion 33 through the puncture slit 331 into the leading hole 34.

With reference to FIGS. 1 to 3, the pushing module 40 is mounted in the medication storage space 23 through the pushing module entrance 22, and can be pushed or pulled along the axis of the protecting tube 10. The pushing module 40 comprises a pushing stick 41 and a pushing plug 42. The pushing stick 41 is mounted on the pushing plug 42 in the medication storage space 23 through the pushing module entrance 22 and comprises a combining portion 411 and a handle portion 412. The combining portion 411 is mounted on a front end of the pushing stick 41 and is located in the medication storage space 23. The handle portion 412 is mounted on a rear end of the pushing stick 41 for facilitating pushing or pulling of the pushing stick 41. The pushing plug 42 may be made of chemically inert thermopalsctic elastomer. The pushing plug 42 is removably mounted on the combining portion 411 by threads, and is slidably and hermetically mounted in the medication storage space 23.

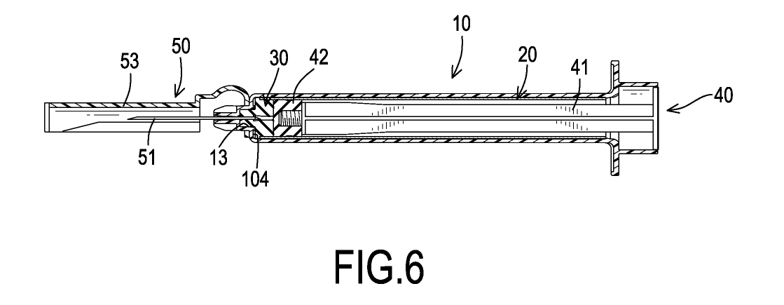

With reference to FIGS. 1 to 3, the needle module 50 is mounted on the needle mounting portion 11 and comprises a needle body 51, a protecting cover 52, and a safety cover 53. The needle body 51 comprises a puncture end 512 and an injection end 511. The puncture end 512 is fixed in the needle mounting hole 110 and penetrates the protrusion 33 through the puncture slit 331 into the leading hole 34, such that the needle body 51 can communicate with the leading hole 34 and the medication storage space 23. The protecting cover 52 is mounted around the needle mounting portion 11 and comprises a sealing plug 521 mounted in the protecting cover 52. The sealing plug 521 is made of chemically inert thermoplastic elastomer and is transfixed by the injection end 511. The safety cover 53 is mounted around the protecting tube 10 and is selectively engaged with the needle body 51 after the injection of the medication, as shown in FIG. 6, to keep the user from touching the needle body 51 inadvertently.

With reference to FIG. 2, under an unpacked condition, the medication storage space 23 is pre-filled with medication. It is noted that the medication can be stored in the closed medication storage space 23 safely and stably since the medication filling tube 20 is made of stable glass or chemically inert polymers, such as cyclic olefin, and is closed by the chemically inert separating plug 30 and the pushing plug 42. Moreover, the medication filling tube 20 is protected by the surrounding protecting tube 10. The separating plug 30 is transfixed by the punctured end 512 of the needle body 51 to guide the medication. The injection end 511 of the needle body 51 is sealed by the chemically inert sealing plug 521. When the disposable pre-filled syringe is in use, the protecting cover 52 is removed from the needle body 51. The pushing stick 41 is engaged with the pushing plug 42 by threads. The pushing stick 41 is further pushed by the user to push the medication. The medication stored in the medication storage space 23 is exerted with the pressure from the pushing plug 42, and then flows into the needle body 51 for injection on a patient. With reference to FIG. 6, after the injection is finished, the safety cover 53 covers the needle body 51 to keep the user from touching the needle body 51.

The disposable pre-filled syringe not only can simplify the medication preparing process in the clinical medical practice, but also can reduce medical waste pollution and erroneous dosing in the medication administration as compared with the conventional syringes use. The medication filling tube 20 can store medication on a long term basis to decrease the amount of discarded conventional medication containers. Furthermore, the disposable pre-filled syringe can be adopted for immediate use to increase the efficiency of clinical operation.

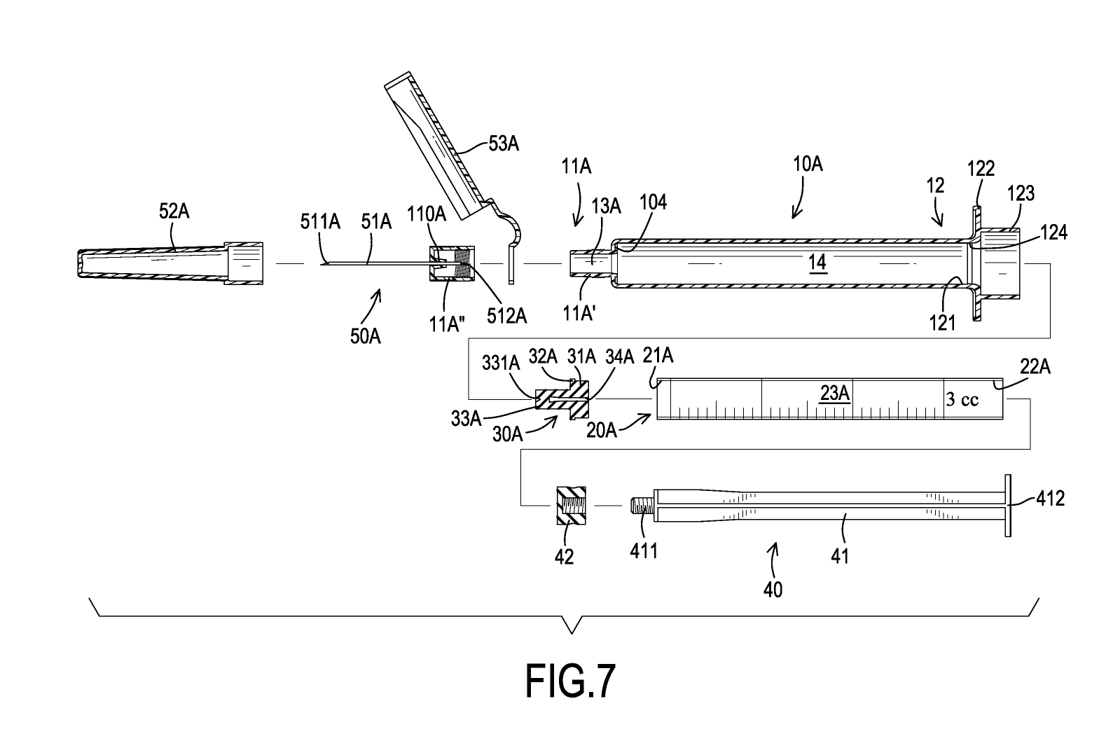

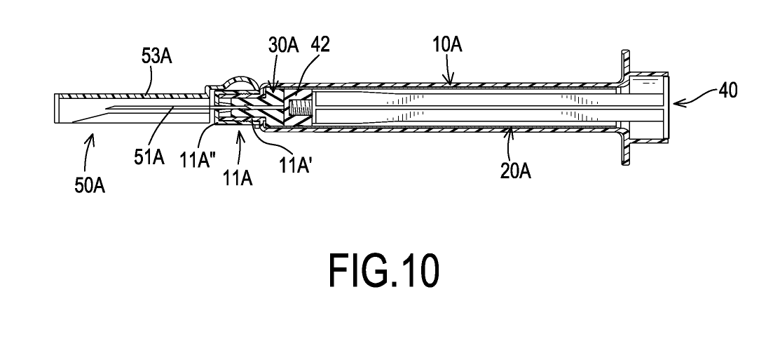

With reference to FIGS. 7 to 10, a second preferred embodiment of the disposable pre-filled syringe in accordance with the present invention is similar with the first preferred embodiment and comprises a protecting tube 10A, a medication filling tube 20A, a separating plug 30A, a pushing module 40 and a needle module 50A.

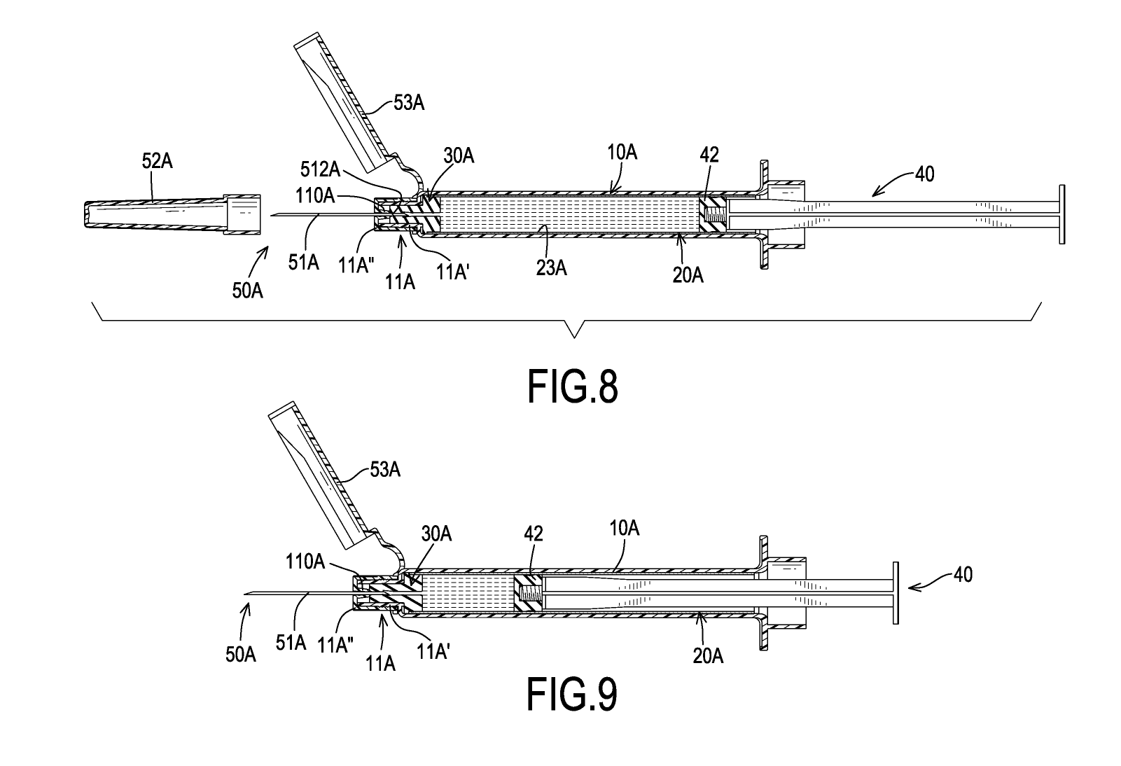

With reference to FIGS. 7 and 8, the protecting tube 10A comprises a needle mounting portion 11A mounted on a front end of the protecting tube 10A. The needle mounting portion 11A is mounted on the front end of the protecting tube 10A and comprises a fixing portion 11A′, a fixing plug cover 11A″ and a needle mounting hole 110A. The fixing portion 11A′ is mounted on the front end of the protecting tube 10A, comprises an outer thread and a trough 13A extending through the fixing portion 11A′ and communicating with the tube space 14 of the protecting tube 10A. The fixing plug cover 11A″ is combined with the fixing portion 11A′ and comprises an inner thread. The fixing plug cover 11A″ and the fixing portion 11A′ are combined with each other by threads. The needle mounting hole 110A is formed in the fixing plug cover 11A″ and can be penetrated through by a needle.

The medication filling tube 20A is mounted in the protecting tube 10A and comprises a medication storage space 23A, a plug hole 21A, and a pushing module entrance 22A. The separating plug 30A is mounted in the medication filling tube 20A, abuts between the protecting tube 10A, and the medication filling tube 20A, and has a leading hole 34A formed in the separating plug. The pushing module 40 is inserted into the medication filling tube 20A that is mounted in the protecting tube 10A.

The needle module 50A is mounted on the front end of the needle mounting portion 11A and comprises a needle body 51A, a protecting cover 52A, and a safety cover 53A. The needle body 51A is fixed in the needle mounting hole 110A, protrudes through the fixing plug cover 11A″, and comprises a frontward injection end 511A and a rearward puncture end 512A.

With reference to FIGS. 8 to 10, the medication filling tube 20A is pre-filled with medication prior to use. The fixing plug cover 11A″ is engaged with the fixing portion 11A′ by threads, and the needle body 51A fixed in the needle mounting hole 110A of the fixing plug cover 11A″ pierces through the separating plug 30A to communicate with the leading hole 34A and the medication storage space 23A, such that the needle module 50A can be fixed relative to the protecting tube 10A. In use, the protecting cover 52A is removed to expose the injection end 511A of the needle body 51A. After the injection is finished, the safety cover 53A covers the needle body 51A to keep the user from touching the needle body 51A.

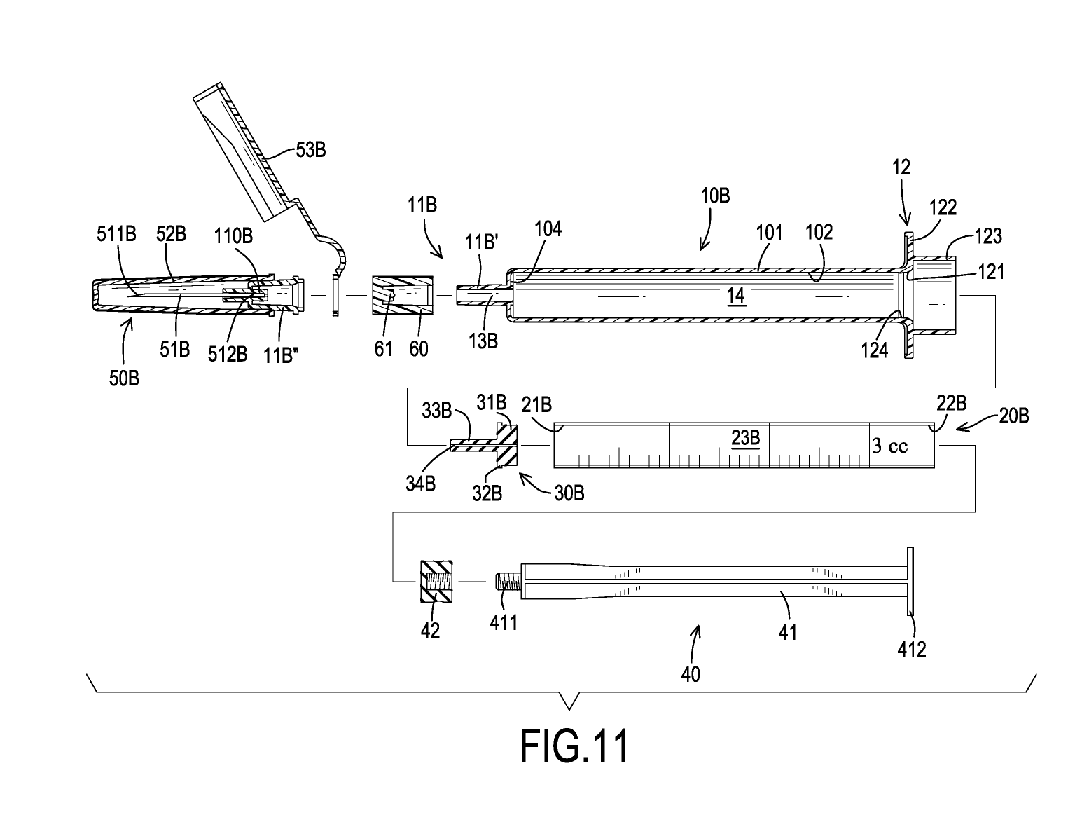

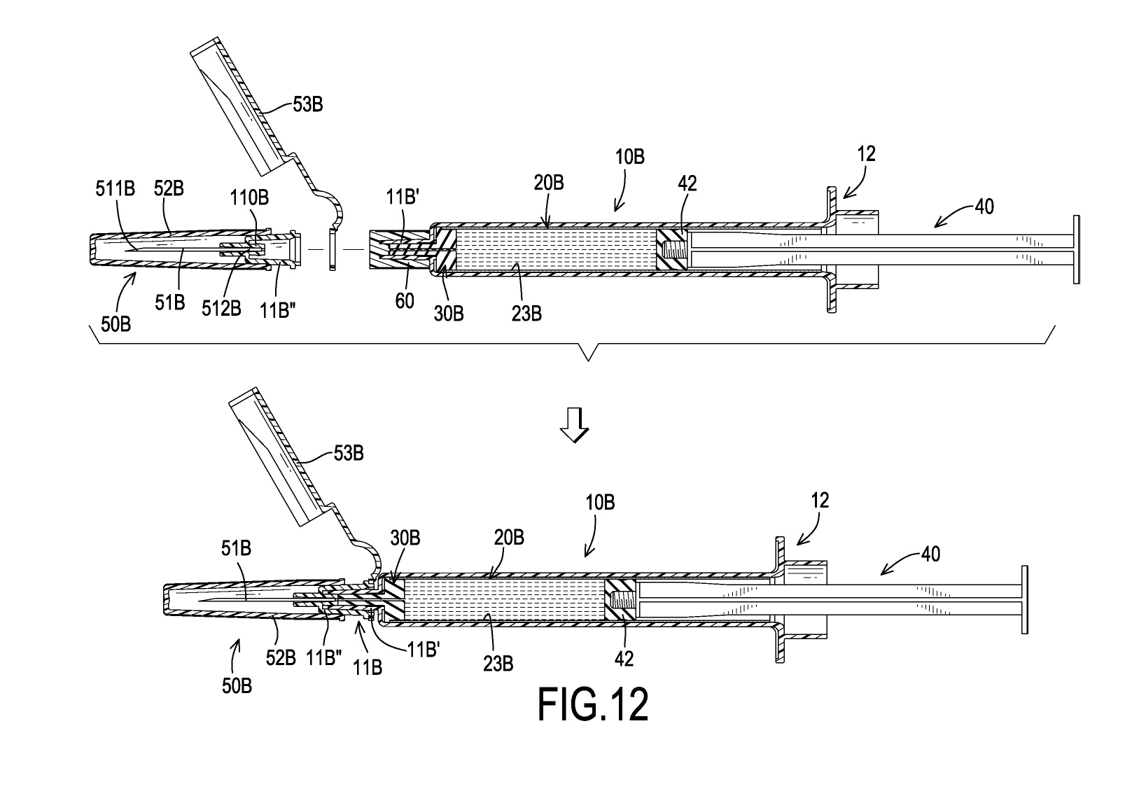

With reference to FIGS. 11 and 12, a third preferred embodiment of the disposable pre-filled syringe in accordance with the present invention comprises a protecting tube 10B, a medication filling tube 20B, a separating plug 30B, a pushing module 40, a needle module 50B and an airtight plug 60.

The protecting tube 10B comprises a needle mounting portion 11B. The needle mounting portion 11B comprises a fixing portion 11B′, a fixing plug cover 11B″ and a needle mounting hole 110B. The fixing plug cover 11B″ is selectively mounted around the fixing portion 11B′. The needle mounting hole 110B is formed through the fixing plug cover 11B″.

The medication filling tube 20B is mounted in the protecting tube 10B. The separating plug 30B is mounted in medication filling tube 20B, abuts between the medication filling tube 20B and the protecting tube 10B, and has a leading hole 34B formed through the separating plug 30B. An elongated protrusion 33B of the separating plug 30B is inserted into a trough 13B of the protecting tube 10B. The leading hole 34B of the separating plug 30B is formed through the protrusion 33B and communicates with a medication storage space 23B of the medication filling tube 20B.

The needle module 50B is removably and selectively mounted on the fixing plug cover 11B″ and comprises a needle body 51B, a protecting cover 52B, and a safety cover 53B. The needle body 51B is fixed in the needle mounting hole 110B and comprises a frontward injection end 511B and a rearward fixing end 512B. The protecting cover 52B and the safety cover 53B are mounted around the fixing plug cover 11B″.

The airtight plug 60 is removably and selectively mounted around the fixing portion 11B′ and comprises a protruding block 61. The protruding block 61 protrudes from the airtight plug 60, is located in the airtight plug 60, is selectively inserted into the fixing portion 11B′ and seals the leading hole 34B to keep the pre-filled medication in the medication storage space 23B from leakage.

With reference to FIG. 12, prior to use, the airtight plug 60 is removed from the fixing portion 11B′, and then the fixing plug cover 11B″ is mounted around the fixing portion 11B′. The protecting cover 52B is further removed for giving an injection on a patient by the needle body 51B.

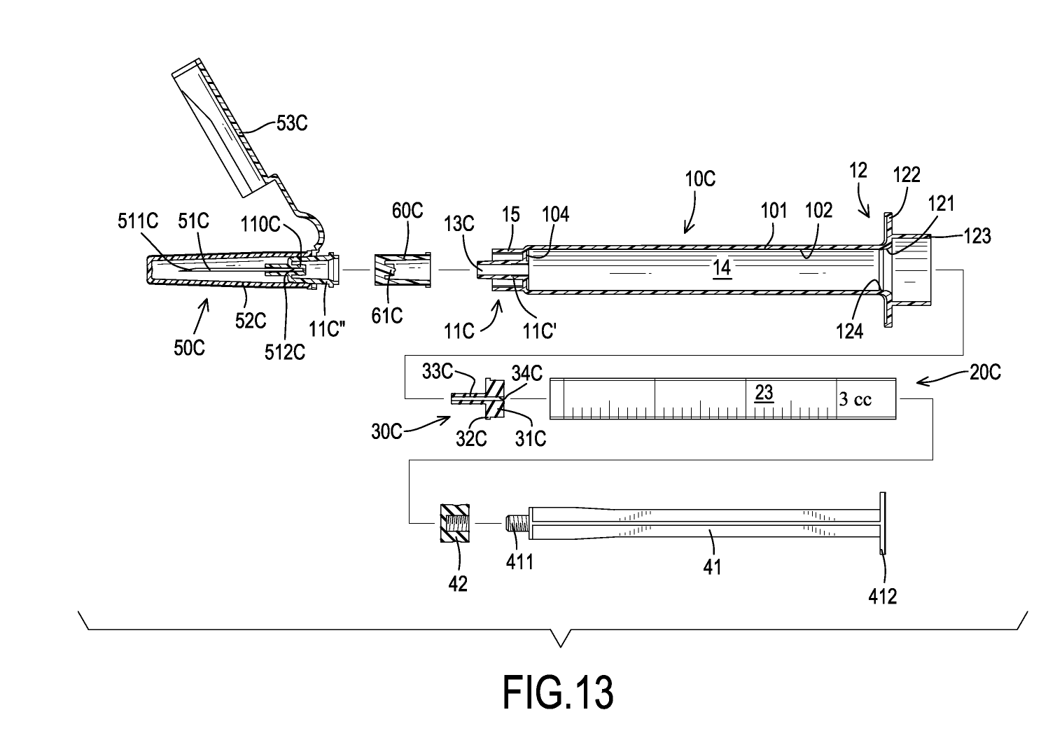

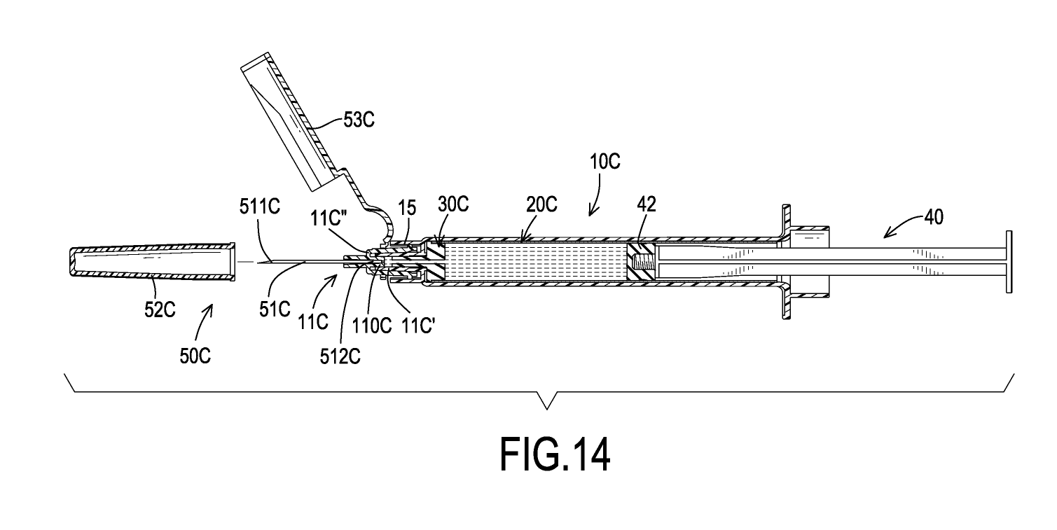

With reference to FIGS. 13 and 14, a fourth preferred embodiment of the disposable pre-filled syringe in accordance with the present invention comprises a protecting tube 10C, a medication filling tube 20C, a separating plug 30C, a pushing module 40, a needle module 50C, and an airtight plug 60C.

The protecting tube 10C comprises a needle mounting portion 11C and an auxiliary fixed portion 15. The needle mounting portion 11C comprises a fixing portion 11C′, a fixing plug cover 11C″ and a needle mounting hole 110C. The fixing plug cover 11C″ is selectively mounted around the fixing portion 11C′. The needle mounting hole 110C is formed through the fixing plug cover 11C″. The auxiliary fixed portion 15 surrounds the needle mounting portion 11C to assist in fixing the fixing plug cover 11C″. The medication filling tube 20C comprises a medication storage space 23C formed through the medication filling tube 20C.

The separating plug 30C is mounted in the medication filling tube 20C, abuts between the protecting tube 10C and the medication filling tube 20C, and has a leading hole 34C formed through the separating plug 30C. The needle module 50C is removably and selectively mounted on the fixing plug cover 11C″ and comprises a needle body 51C, a protecting cover 52C, and a safety cover 53C. The needle body 51C is fixed in the needle mounting hole 110C and comprises a frontward injection end 511C and a rearward fixing end 512C. The protecting cover 52C is mounted around the fixing plug cover 11C″. The safety cover 53C is engaged with the fixing plug cover 11C″. The airtight plug 60C is removably and selectively mounted around the fixing portion 11C′ and abuts between the fixing portion 11C′ and the auxiliary fixed portion 15. The airtight plug 60C comprises a protruding block 61C. The protruding block 61C protrudes from the airtight plug 60C, is located in the airtight plug 60, is selectively inserted into the fixing portion 11C′ and seals the leading hole 34C while the airtight plug 60C mounted around the fixing portion 11C′. Prior to use, the airtight plug 60C is removed from the fixing portion 11C′, and then the fixing plug cover 11C″ is mounted around the fixing portion 11C′ and is engaged with the auxiliary fixed portion 15. The protecting cover 52C is further removed for giving an injection on a patient by the needle body 51C.

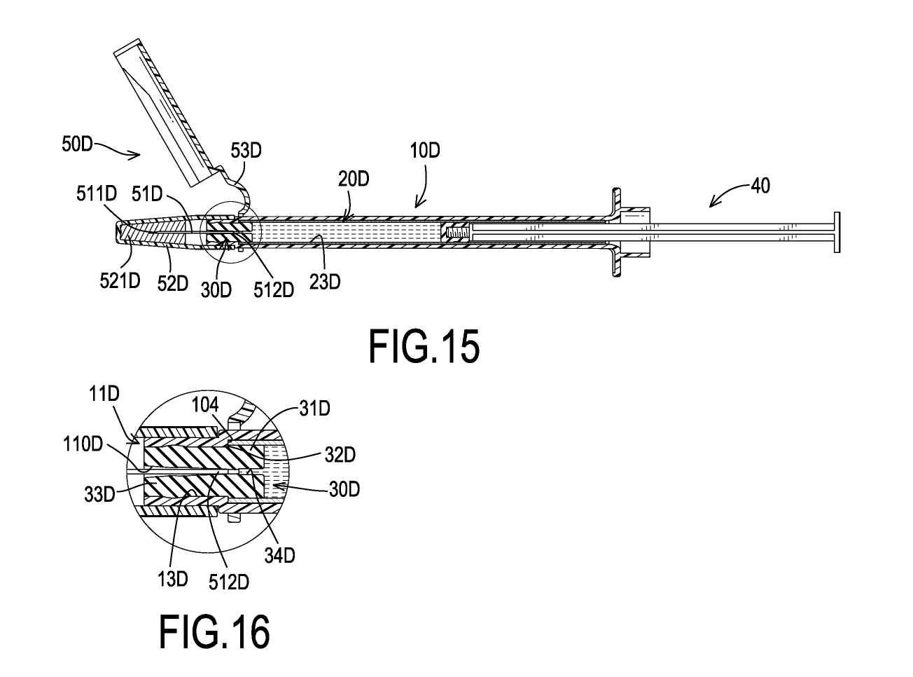

With reference to FIGS. 15 and 16, a fifth preferred embodiment of the disposable pre-filled syringe in accordance with the present invention comprises a protecting tube 10D, a needle mounting portion 11D, a medication filling tube 20D, a separating plug 30D, a pushing module 40, and a needle module 50D. The capacities of the protecting tube 10D and the medication filling tube 20D may both be featured for the medication amount of about 1 cubic centimeter (cc) in volume.

The separating plug 30D is mounted in the protecting tube 10D and the medication filling tube 20D and has a leading hole 34D formed through a protrusion 33D of the separating plug 30D. The needle mounting portion 11D comprises a needle mounting hole 110D formed through the protrusion 33D of the separating plug 30D along the axis of the protecting tube 10D and communicates with the medication filling tube 20D.

The needle module 50D is mounted on the front end of the needle mounting portion 11D and comprises a needle body 51D, a protecting cover 52D, and a safety cover 53D. The needle body 51D is fixed in the needle mounting hole 110D, communicates with leading hole 34D and the medication storage space 23D of the medication filling tube 20D, and comprises a frontward injection end 511D and a rearward fixing end 512D.

The protecting cover 52D is mounted around the needle mounting portion 11D and comprises a sealing plug 521D mounted in the protecting cover 52D. The sealing plug 521D is made of chemically inert thermoplastic elastomer and is transfixed by the injection end 511D. The safety cover 53D is mounted around the protecting tube 10D and is selectively engaged with the needle body 51D after the injection of the medication to keep the user from touching the needle body 51D inadvertently.

A needle body 51D of the needle module 50D is inserted into a sealing plug 521D of a protecting cover 52D of the needle module 50D, and is inserted into a leading hole 34D of the separating plug 30D to communicate with a drug mounting space 23D of the medication filling tube 20D.

A protrusion 33D of the separating plug 30D is mounted into a trough 13D of the protecting tube 10D. A block flange 32D of the separating plug 30D abuts the abutting portion 104 and the medication filling tube 20D.

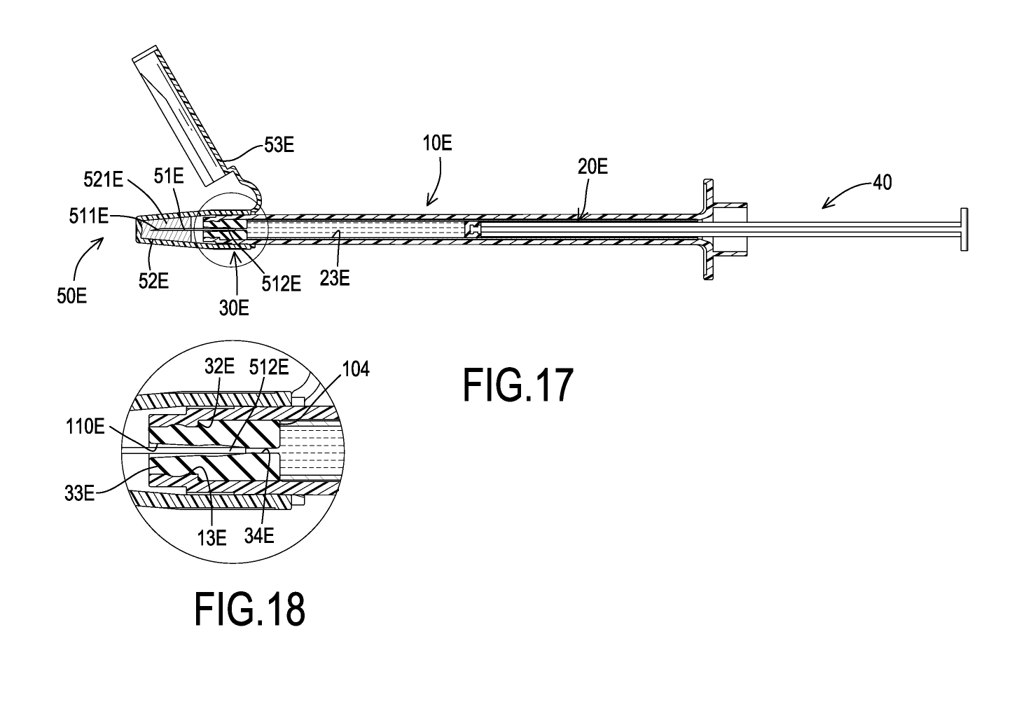

With reference to FIGS. 17 and 18, a sixth preferred embodiment of the disposable pre-filled syringe in accordance with the present invention comprises a protecting tube 10E, a needle mounting portion 11E, a medication filling tube 20E, a separating plug 30E, a pushing module 40, and a needle module 50E. The capacities of the protecting tube 10E and the medication filling tube 20E may both be featured for the medication amount of about 0.5 cc in volume.

The separating plug 30E is mounted in the protecting tube 10E and the medication filling tube 20E and has a leading hole 34E. The needle mounting portion 11E comprises a needle mounting hole 110E formed through the protrusion 33E of the separating plug 30E along the axis of the protecting tube 10E and communicates with the medication filling tube 20E.

It is noted that the technical and structural features of the sixth preferred embodiment of the disposable pre-filled syringe in accordance with the present invention are substantially similar to the fifth preferred embodiment of the disposable pre-filled syringe in accordance with the present invention. What the differences are the sizes of the related components.

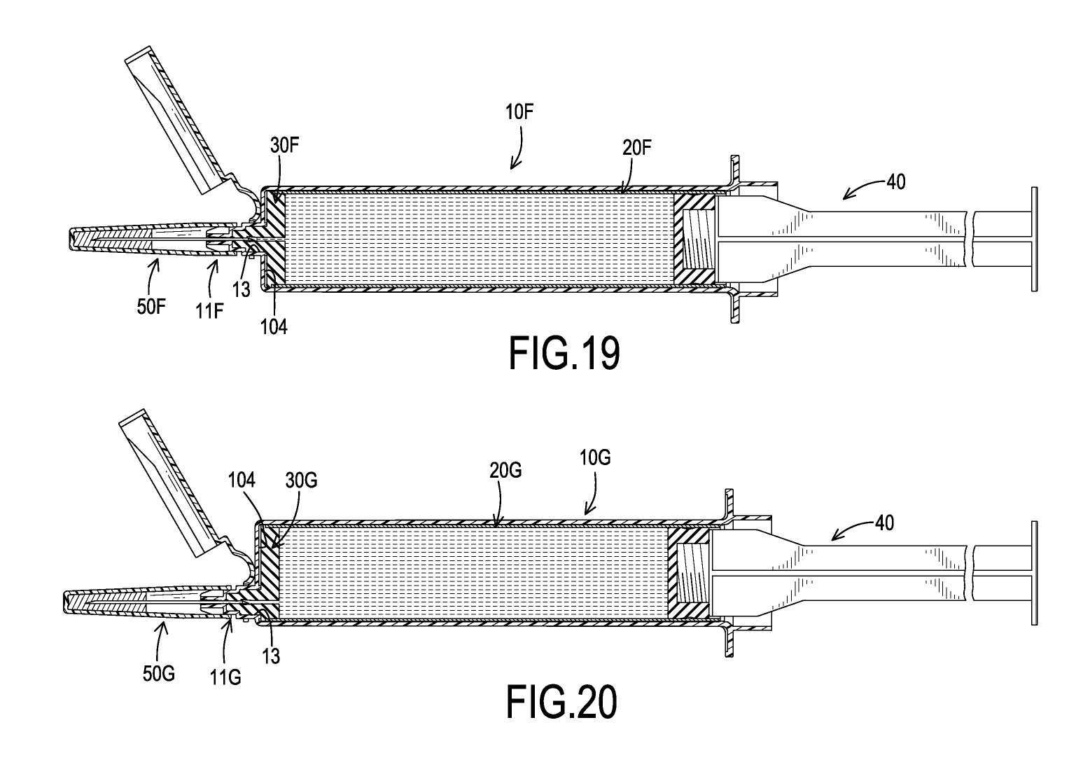

With reference to FIG. 19, a seventh preferred embodiment of the disposable pre-filled syringe in accordance with the present invention comprises a protecting tube 10F, a medication filling tube 20F, a separating plug 30F, a pushing module 40, and a needle module 50F. The capacities of the protecting tube 10F and the medication filling tube 20F may both be featured for the medication amount of about 25 cc in volume. A needle mounting portion 11F of the protecting tube 10F is located at the middle of a front end of the protecting tube 10F.

With reference to FIG. 20, an eighth preferred embodiment of the disposable pre-filled syringe in accordance with the present invention comprises a protecting tube 10G, a medication filling tube 20G, a separating plug 30G, a pushing module 40, and a needle module 50G. The capacities of the protecting tube 10G and the medication filling tube 20G may both be featured for the medication amount of about 25 cc in volume. A needle mounting portion 11G of the protecting tube 10G is located at a peripheral of a front end of the protecting tube 10G.

Accordingly, the disposable pre-filled syringe uses multiple protecting tubes 10 and multiple medication filling tubes 20 of different sizes to store different volumes of specific medications. The medications are stored in the medication filling tubes 20 safely and stably. The complicated process for clinical medication injection can be simplified efficiently. The risks of medication contamination and erroneous dosing can be reduced. Furthermore, medical waste and discarded containers are also reduced in quantity.

What is claimed is:

1. A disposable pre-filled syringe comprising:
a protecting tube comprising
an axis;
an outside wall annularly formed along the axis of the protecting tube and located at an exterior of the protecting tube;
an inside wall annularly formed along the axis of the protecting tube and located at an interior of the protecting tube;
a holding portion comprising an insertion hole formed through the holding portion;
a needle mounting portion opposite to the holding portion;
a tube space formed in the protecting tube along the axis of the protecting tube and communicating with the insertion hole; and
an abutting portion mounted radially on the inside wall and adjacent to the needle mounting portion;
a medication filling tube being transparent and made of material stable in chemical property, mounted in the tube space and comprising
an axis parallel with the axis of the protecting tube;
a medication storage space formed in the medication filling tube along the axis of the medication filling tube;
a pushing module entrance formed through an end of the medication filling tube and communicating with the medication storage space; and
a plug hole formed through an end of the medication filling tube opposite to the pushing module entrance and communicating with the medication storage space;
a separating plug hermetically mounted in the plug hole and abutting the abutting portion;
a pushing module slidably and hermetically mounted in the medication filling tube and comprising
a pushing stick mounted into the medication storage space through the pushing module entrance and having a combining portion; and
a pushing plug removably mounted on the combining portion and slidably and hermetically mounted in the medication storage space; and
a needle module mounted on the needle mounting portion.

2. The disposable pre-filled syringe as claimed in claim 1, wherein
the holding portion further comprises
a holding flange radially protruding from an exterior of the holding portion; and
an engaging edge circumferentially protruding from an interior of the holding portion and adjacent to the insertion hole; and
the protecting tube further comprises a trough connected with the abutting portion, located at a front end of the protecting tube and communicating with the tube space.

3. The disposable pre-filled syringe as claimed in claim 2, wherein the needle mounting portion further comprises a needle mounting hole formed through the needle mounting portion along the axis of the protecting tube.

4. The disposable pre-filled syringe as claimed in claim 2, wherein the protecting tube further comprises multiple elongated ribs longitudinally protruding from the inside wall along the axis of the protecting tube.

5. The disposable pre-filled syringe as claimed in claim 1, wherein the separating plug further comprises:
a plug body mounted in the plug hole hermetically; and
a block flange annularly formed around the plug body and abutting the abutting portion.

6. The disposable pre-filled syringe as claimed in claim 2, wherein the separating plug further comprises
a plug body mounted in the plug hole hermetically; and
a block flange annularly formed around the plug body and abutting the abutting portion; and
a protrusion formed on an end of the separating plug and mounted in the trough.

7. The disposable pre-filled syringe as claimed in claim 5, wherein the separating plug further comprises a leading hole and communicating with the medication storage space.

8. The disposable pre-filled syringe as claimed in claim 6, wherein the separating plug further comprises a leading hole and communicating with the medication storage space.

9. The disposable pre-filled syringe as claimed in claim 1, wherein the needle module is fixed on the needle mounting portion and comprises a needle body which is fixed in the needle mounting portion, piercing through the separating plug and communicating with the medication storage space.

10. The disposable pre-filled syringe as claimed in claim 2, wherein
the needle module comprises a needle body;
the trough extends through the front end of the protecting tube; and
the separating plug further comprises
an elongated protrusion formed on an end of the separating plug and mounted in the trough; and
a leading hole formed in the separating plug.

11. The disposable pre-filled syringe as claimed in claim 10, wherein the needle mounting portion comprises:
a fixing portion; and
a fixing plug cover engaged with the fixing portion, wherein the needle body fixed in and pierces into the fixing plug cover and the separating plug.

12. The disposable pre-filled syringe as claimed in claim 10, wherein the needle mounting portion comprises:
a fixing portion comprising an outer thread; and
a fixing plug cover comprising an inner thread and engaged with the fixing portion by the outer thread and the inner thread, wherein the needle body fixed in and pierces into the fixing plug cover and the separating plug.

13. The disposable pre-filled syringe as claimed in claim 2, wherein
the needle module comprises a needle body;
the trough extends through the front end of the protecting tube; and
the separating plug further comprises
an elongated protrusion formed on an end of the separating plug and mounted in the trough; and
a leading hole formed through the separating plug.

14. The disposable pre-filled syringe as claimed in claim 13, wherein the needle mounting portion comprises:
a fixing portion; and
a fixing plug cover engaged with the fixing portion, wherein the needle body pierces into and fixed on the fixing plug cover.

15. The disposable pre-filled syringe as claimed in claim 13, wherein
the protecting tube further comprises an auxiliary fixed portion surrounding the needle mounting portion;
the needle mounting portion comprises
a fixing portion; and
a fixing plug cover engaged with the fixing portion and the auxiliary fixed portion, wherein the needle body pierces into and fixed on the fixing plug cover.

16. The disposable pre-filled syringe as claimed in claim 13, wherein the needle body is fixed in and communicates with leading hole

17. The disposable pre-filled syringe as claimed in claim 8, wherein the needle mounting portion of the protecting tube is located at a middle of a front end of the protecting tube.

18. The disposable pre-filled syringe as claimed in claim 8, wherein the needle mounting portion of the protecting tube is located at a peripheral of a front end of the protecting tube.

19. The disposable pre-filled syringe as claimed in claim 1, wherein the medication filling tube is made of glass.

20. The disposable pre-filled syringe as claimed in claim 1, wherein the medication filling tube is made of cyclic olefin polymers.

21. The disposable pre-filled syringe as claimed in claim 2, wherein the medication filling tube is made of glass.

22. The disposable pre-filled syringe as claimed in claim 2, wherein the medication filling tube is made of cyclic olefin polymers.

23. The disposable pre-filled syringe as claimed in claim 3, wherein the medication filling tube is made of glass.

24. The disposable pre-filled syringe as claimed in claim 3, wherein the medication filling tube is made of cyclic olefin polymers.

25. The disposable pre-filled syringe as claimed in claim 4, wherein the medication filling tube is made of glass.

26. The disposable pre-filled syringe as claimed in claim 4, wherein the medication filling tube is made of cyclic olefin polymers.

27. The disposable pre-filled syringe as claimed in claim 7, wherein the medication filling tube is made of glass.

28. The disposable pre-filled syringe as claimed in claim 7, wherein the medication filling tube is made of cyclic olefin polymers.

29. The disposable pre-filled syringe as claimed in claim 9, wherein the medication filling tube is made of glass.

30. The disposable pre-filled syringe as claimed in claim 9, wherein the medication filling tube is made of cyclic olefin polymers.

31. A disposable pre-filled syringe comprising:
a protecting tube comprising
an axis;
an outside wall annularly formed along the axis of the protecting tube and located at an exterior of the protecting tube;
an inside wall annularly formed along the axis of the protecting tube and located at an interior of the protecting tube;
a holding portion comprising an insertion hole formed through the holding portion;
a needle mounting portion opposite to the holding portion;
a tube space formed in the protecting tube along the axis of the protecting tube and communicating with the insertion hole; and
an abutting portion mounted radially on the inside wall and adjacent to the needle mounting portion;
a trough connected with the abutting portion, formed through a front end of the protecting tube and communicating with the tube space.
a medication filling tube being transparent and made of material stable in chemical property, mounted in the tube space and comprising
an axis parallel with the axis of the protecting tube;
a medication storage space formed in the medication filling tube along the axis of the medication filling tube;
a pushing module entrance formed through an end of the medication filling tube and communicating with the medication storage space; and
a plug hole formed through an end of the medication filling tube opposite to the pushing module entrance and communicating with the medication storage space;
a separating plug hermetically mounted in the plug hole and abutting the abutting portion and the medication filling tube, comprising a protrusion mounted in the trough and a leading hole formed in the separating plug; and
a pushing module slidably and hermetically mounted in the medication filling tube and comprising
a pushing stick mounted into the medication storage space through the pushing module entrance and having a combining portion; and
a pushing plug removably mounted on the combining portion and slidably and hermetically mounted in the medication storage space.

32. A disposable pre-filled syringe comprising:
a protecting tube comprising
an axis;
an outside wall annularly formed along the axis of the protecting tube and located at an exterior of the protecting tube;
an inside wall annularly formed along the axis of the protecting tube and located at an interior of the protecting tube;
a holding portion comprising an insertion hole formed through the holding portion;
a needle mounting portion opposite to the holding portion;
a tube space formed in the protecting tube along the axis of the protecting tube and communicating with the insertion hole; and
an abutting portion mounted radially on the inside wall and adjacent to the needle mounting portion;
a medication filling tube being transparent and made of material stable in chemical property, mounted in the tube space and comprising
an axis parallel with the axis of the protecting tube;
a medication storage space formed in the medication filling tube along the axis of the medication filling tube;
a pushing module entrance formed through an end of the medication filling tube and communicating with the medication storage space; and
a plug hole formed through an end of the medication filling tube opposite to the pushing module entrance and communicating with the medication storage space;
a separating plug hermetically mounted in the plug hole and abutting the abutting portion; and
a pushing module slidably and hermetically mounted in the medication filling tube and comprising
a pushing stick mounted into the medication storage space through the pushing module entrance and having a combining portion; and
a pushing plug removably mounted on the combining portion and slidably and hermetically mounted in the medication storage space.

33. A disposable pre-filled syringe comprising:
a protecting tube comprising
an axis;
an outside wall annularly formed along the axis of the protecting tube and located at an exterior of the protecting tube;
an inside wall annularly formed along the axis of the protecting tube and located at an interior of the protecting tube;
a holding portion comprising an insertion hole formed through the holding portion;
a needle mounting portion opposite to the holding portion;
a tube space formed in the protecting tube along the axis of the protecting tube and communicating with the insertion hole; and
an abutting portion mounted radially on the inside wall and adjacent to the needle mounting portion;
a trough connected with the abutting portion, formed through a front end of the protecting tube and communicating with the tube space;
an airtight plug removably mounted around the needle mounting portion and comprising a protruding block protruding from and located in the airtight plug;
a medication filling tube being transparent and made of material stable in chemical property, mounted in the tube space and comprising
an axis parallel with the axis of the protecting tube;
a medication storage space formed in the medication filling tube along the axis of the medication filling tube;
a pushing module entrance formed through an end of the medication filling tube and communicating with the medication storage space; and
a plug hole formed through an end of the medication filling tube opposite to the pushing module entrance and communicating with the medication storage space;
a separating plug hermetically mounted into the plug hole and abutting the abutting portion and the medication filling tube, comprising a protrusion mounted in the trough and a leading hole formed through the separating plug; and
a pushing module slidably and hermetically mounted in the medication filling tube and comprising
a pushing stick mounted into the medication storage space through the pushing module entrance and having a combining portion; and
a pushing plug removably mounted on the combining portion and slidably and hermetically mounted in the medication storage space.
